(19) United States
(12) Patent Application Publication
(10) Pub. No.: US 2005/0123477 A1
(43) Pub. Date: Jun. 9, 2005

(54) Benzothiazole derivatives for in vivo imaging of amloid plaques

(76) Inventors: Ian Wilson, Buckinghamshire (GB); Sajinder Kaur Luthra, London (GB); Frank Brady, London (GB)

Correspondence Address: AMERSHAM HEALTH, IP DEPARTMENT, 101 CARNEGIE CENTER, PRINCETON, NJ, 08540-6231, US

(21) Appl. No.: 10/504,231

(22) PCT Filed: Feb. 12, 2003
(86) PCT No.: PCT/GB03/00584

(30) Foreign Application Priority Data
Feb. 13, 2002 (GB) 0203391.8
Jul. 31, 2002 (GB) 0217713.7

(51) Int. Cl. A61K 49/00; C07D 417/02

(52) U.S. Cl. 424/9.6; 548/156

(57) ABSTRACT

The invention provides use of a compound of formula (I): or a salt thereof, wherein: R¹ is ¹²⁵I, ¹²⁴I, ¹²³I, ⁷⁵Br, ⁷⁶Br, or ¹⁸F; R² is C₁₋₆ alkyl; and R³ is selected from hydrogen, C₁₋₆ alkyl, —C(O)C₁₋₆ alkyl, —C(O)C₁₋₆ haloalkyl, and —C(O)CH(R⁴)NH₂; wherein R⁴ is selected from hydrogen, C₁₋₆alkyl, C₁₋₆hydroxyalkyl, and C₁₋₆aminoalkyl; for the manufacture of a radiopharmaceutical for the in vivo diagnosis or imaging of an amyloid-associated disease, particularly Alzheimer's disease.

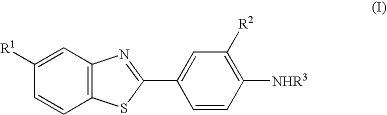

The present invention relates to the field of diagnostic imaging of Alzheimer's disease and provides compounds useful in such diagnostic imaging.

Alzheimer's disease is the fourth most common cause of death in the western world, after heart disease, cancer and strokes. In the USA there are approximately 4 million people suffering with Alzheimer's disease, at an annual cost of $100 billion. Therefore, the cost per person in the USA is $25,000 per year. There are currently 20 million sufferers of dementia in the world. This is set to double to 40 million by the year 2025 as the number of people aged 65 doubles from 390 million now to 800 million in 2025. Of these 40 million, approximately 56 percent will be suffering from Alzheimer's disease, accounting for 22.2 million.

The in vivo imaging techniques used at present do not in all cases differentiate the diagnosis of Alzheimer's disease from other forms of dementia. The differential diagnosis of patients will become increasingly important as more treatments become available. Imaging agents will also be required to image Alzheimer patients at earlier stages of the disease to allow preventive treatment, and for monitoring disease progression

Currently the only definitive test for Alzheimer's disease is examination of the brain at autopsy for the presence of distinctive pathophysiologies. One of the most widely acknowledged of these pathophysiologies is the presence of senile plaques in brain tissue. Senile plaques are deposits of a 40-43 amino acid protein called the β-amyloid protein. They are an early and invariant aspect of the disease and it is thought that the deposition of β-amyloid occurs some time prior to the onset of clinical symptoms.

Amyloid-specific radiotracers have been suggested as potential imaging agents for Alzheimer's disease. Congo Red has been demonstrated to be an effective binder of β-amyloid, but does not cross the blood-brain barrier (BBB) well (Klunk et al 1994 Neurobiology of Aging Vol. 15 pp. 691-698). There is no convincing functional evidence that abnormalities in the BBB reliably exist in Alzheimer's (Kalaria 1992, Cerebrovascular and Brain Metabolism Reviews, Vol 4, p 226). Therefore, an important property of an Alzheimer's imaging agent is that it crosses the BBB.

WO01/14354 describes a broad class of substituted 2-arylbenzazole compounds and their use as anti-tumour agents.

The aim of the present invention is the provision of novel agents for imaging Alzheimer's disease. To be able to successfully image Alzheimer's disease, an agent must be capable of crossing the BBB as well as binding to β-amyloid.

In a first aspect, this invention provides use of a compound of formula (I): 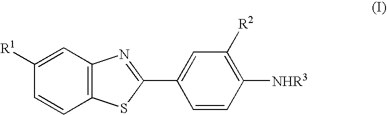
or a salt thereof, wherein:
R¹ is ¹²⁵I, ¹²⁴I, ¹²³I, ⁷⁵Br, ⁷⁶Br or ¹⁸F;
R² is C₁₋₆ alkyl; and
R³ is selected from hydrogen, C₁₋₆ alkyl, —C(O)C₁₋₆ alkyl, —C(O)C₁₋₆ haloalkyl, and —C(O)CH(R⁴)NH₂;
   wherein R⁴ is selected from hydrogen, C₁₋₆alkyl, C₁₋₆hydroxyalkyl, and C₁₋₆aminoalkyl;
for the manufacture of a radiopharmaceutical for the in vivo diagnosis or imaging of an amyloid-associated disease, particularly Alzheimer's disease.

In a particular aspect, this invention provides use of a compound of formula (I): 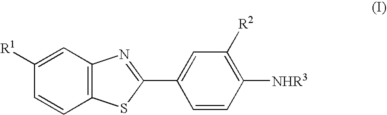
or a salt thereof, wherein:
R¹ is ¹²⁵I, ¹²⁴I, ¹²³I, ⁷⁵Br, ⁷⁶Br, or ¹⁸F;
R² is C₁₋₆ alkyl; and
R³ is selected from hydrogen, C₁₋₆ alkyl, —C(O)C₁₋₆ alkyl, and —C(O)C₁₋₆ haloalkyl;
for the manufacture of a radiopharmaceutical for the in vivo diagnosis or imaging of an amyloid-associated disease, particularly Alzheimer's disease.

In the alterative, there is provided a method for the in vivo diagnosis or imaging of amyloid-associated disease in a subject (preferably, a human) comprising administration of a compound of formula (I) or a salt thereof. The method is especially preferred for the in vivo diagnosis and imaging of Alzheimer's disease.

“Amyloid-associated” diseases include Alzheimer's disease, familial Alzheimer's disease, type II diabetes, Down's syndrome, homozygotes for the apolipoprotein E4 allele, rheumatoid arthritis, systemic amyloidosis (primary and secondary), and haemorrhagic stroke.

“Alkyl” used either alone or as part of another group (such as haloalkyl) is defined herein as any straight or branched CₙH₂ₙ₊₁ group, wherein unless otherwise specified n is 1 to 6.

The term “halo” means a group selected from fluoro, chloro, bromo, and iodo.

Suitable salts of the compounds of formula (I) include acid addition salts such as those derived from mineral acids, such as hydrochloric, hydrobromic, phosphoric, metaphosphoric, nitric, and sulphuric acids or those derived from organic acids such as tartaric, acetic, trifluoroacetic, citric, malic, lactic, fumaric, benzoic, glycollic, glucbnic, succinic, methanesulphonic, and arylsulphonic (for example para-toluenesulphonic) acids.

In a further aspect of the present invention, there is provided a compound of formula (I) or a salt thereof, for in vivo diagnosis or imaging of amyloid-associated diseases, preferably of Alzheimer's disease.

A compound of formula (I) or a salt thereof is preferably administered in a radiopharmaceutical formulation comprising the compound of the invention. A “radiopharmaceutical formulation” is defined in the present invention as a formulation comprising compound of formula (I) or a salt thereof in a form suitable for administration to humans, preferably a radiopharmaceutical formulation further comprises a physiologically acceptable excipient. Administration is preferably carried out by injection of the formulation as an aqueous solution. Such a formulation may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid). The dose of a compound of formula (I) or a salt thereof will vary depending on the exact compound to be administered, the weight of the patient, and other variables as would be apparent to a physician skilled in the art. Generally, the dose would lie in the range 0.001 μg/kg to 10 μg/kg, preferably 0.01 μg/kg to 1.0 μg/kg.

In a particular aspect of the invention, in the compound of formula (I), R¹ is ¹²³I. Such compounds are useful for SPECT imaging of amyloid-associated diseases, such as Alzheimer's disease.

In another particular aspect of the invention, in the compound of formula (I), R¹ is ¹²⁵I. Such compounds are useful for SPECT imaging of amyloid-associated diseases, such as Alzheimer's disease.

In another particular aspect of the invention, in the compound of formula (I), R¹ is ¹⁸F. Such compounds are useful for Positron Emission Tomography (PET) imaging of amyloid-associated diseases, such as Alzheimer's disease.

In another particular aspect of the invention, in the compound of formula (I), R¹ is ¹²⁴I. Such compounds are useful for PET imaging of amyloid-associated diseases, such as Alzheimer's disease.

Preferred compounds of formula (I) include those in which:
R¹ is ¹²⁵I, ¹²⁴I, ¹²³I or ¹⁸F;
R² is methyl; and
R³ is selected from hydrogen and —C(O)C₁₋₆ haloalkyl, suitably —C(O)C₁₋₆ fluoroalkyl, most suitably —C(O)CF₃.

Where R³ is —C(O)CH(R⁴)NH₂, R⁴ is preferably C₁₋₆aminoalkyl, and is more preferably —(CH₂)₄NH₂. One such compound of particular interest is 5-[¹⁸F-fluoro-2-(4′-amino-3′-methylphenyl)benzothiazole lysyl amide or a salt thereof such as the dihydrochloride salt.

Particularly preferred compounds of formula (I) include:
5-[¹²⁵I]-iodo-2-(4′-amino-3′-methylphenyl)benzothiazole;
5-[¹²⁵I]-iodo-2-(4′-trifluoromethylamido-3′-methylphenyl)benzothiazole;
5-[¹⁸F]-fluoro-2-(4′-amino-3′-methylphenyl)benzothiazole;
5-[¹⁸F]-fluoro-2-(4′-trifluoromethylamido-3′-methylphenyl)benzothiazole;
or a salt thereof.

Certain of the compounds of formula (I) are novel, and therefore, as a separate aspect of the invention there is provided a compound of formula (Ia): 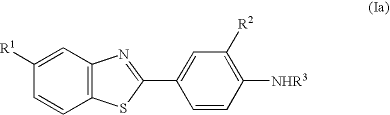
or a salt thereof, wherein:
R¹ is ¹²⁵I, ¹²⁴I, ¹²³I, ⁷⁵Br, or ⁷⁶Br;
R² is C₁₋₆ alkyl; and
R³ is selected from hydrogen, C₁₋₆ alkyl, —C(O)C₁₋₆ alkyl, and —C(O)C₁₋₆ haloalkyl.

Compounds of formula (I) may be prepared by iodination, bromination or fluorination of the corresponding precursor compound in which R¹ is a tri(C₁₋₆alkyl)tin substituent, suitably a trimethyltin substituent. These precursors may be prepared according to the methods described in WO 01/14354 (in particular, Example 44 thereof). The iodination reaction may be effected using an iodide source, such as sodium iodide, in the presence of an oxidising agent, suitably N-chlorosuccinimide, an N-chlorotolylsulphonamide (for example, chloramine T or iodogen), or peracetic acid at non-extreme temperature, preferably at ambient temperature, and in a suitable solvent such as an aqueous buffer at pH 6 to 8, preferably pH 7.4. The fluorination reaction may be effected using the methods described in WO 01/14354 (in particular, Example 45 thereof. The bromination reaction may be effected using a bromide source, such as sodium bromide, in the presence of an oxidising agent, suitably N-chlorosuccinimide, an N-chlorotolylsulphonamide (for example, chloramine T or iodogen), or peracetic acid at non-extreme temperature, preferably at ambient temperature, and in a suitable solvent such as an aqueous buffer.

The [¹⁸F]-fluorinated compounds of formula (I) may also be prepared using the solid-phase fluorination methods described in WO 03/002157 and WO 03/002489.

The invention will now be illustrated by way of the following Examples.

### EXAMPLE 1

### Synthesis of 5-[¹²⁵I]-iodo-2-(4′-amino-3′-methylphenyl)benzothiazole (Compound 1)

The title compound is prepared from Compound 2 (Example 2) by treatment with potassium hydroxide by a method analogous to that described in Example 3.

### EXAMPLE 2

### Synthesis of 5-[¹²⁵I]-iodo-2-(4′-trifluoromethylamido-3′-methylphenyl)benzothiazole (Compound 2)

To 5-trimethylstannyl-2-(4′-trifluoromethylamido-3′-methylphenyl)benzothiazole (which may be prepared as described in WO 01/14354) (100 μg), was added 300 μl sodium phosphate buffer pH7.4, together with 10 mCi [¹²⁵I]-sodium iodide and 100 μl of chloramine T (1 mg/ml in water). This mixture was allowed to react for 30 seconds and the reaction was terminated by adding 100 μl sodium metabisulphite. The mixture was loaded onto a C4 column and separated on reverse phase HPLC using eluents A=water+0.1% trifluoroacetic acid (TFA), B=acetonitrile+0.1% TFA. The product was collected and diluted to 250 μCi/ml in methanol solution and stored at 4° C.

HPLC QC analysis showed the product to have a Radiochemical purity (RCP) of 93% and Iodide content of 0.5%

### EXAMPLE 3

### Synthesis of 5-[¹⁸F]-fluoro-2-(4′-amino-3′-methylphenyl)benzothiazole) (Compound 3)

Fluorine-18, produced as gaseous molecular fluorine (¹⁸F—F) by the ¹⁸O(p, n)¹⁸F nuclear reaction was bubbled through a solution of 5-trimethylstannyl-2-(4′-trifluoromethylamido-3′-methylphenyl)benzothiazole (which may be prepared as described in WO 01/14354) (20 mg, 40 μmol) in acetonitrile (10 mL) and the solvent was removed under reduced pressure. The residue was dissolved in ethanol (1 mL) and potassium hydroxide (1 mL, 0.2 M) and then heated at 80-90° C. for 10 minutes. The resultant mixture was loaded onto an HPLC column (μ-Bondapak C₁₈, 30×0.78 cm i.d.) eluted with a mixture of acetonitrile:water (55:45) at a flow rate of 3 mL min⁻¹. The eluent was monitored for radioactivity and UV absorbance at 254 nm. The radioactive peak having the same retention time of 12-14 min was collected. The eluent was removed under reduced pressure to yield the title compound.

### EXAMPLE 4

### Synthesis of 5-[¹⁸F]-fluoro-2-(4′-trifluoromethylamido-3′-methylphenyl)benzothiazole (Compound 4)

Fluorine-18, either as gaseous molecular fluorine (¹⁸F—F) or [¹⁸F]acetyl hypofluorite is bubbled through a solution of 5-trimethylstannyl-2-(4′-trifluoromethylamido-3′-methylphenyl)benzothiazole (20 mg, 40 μmol) in acetonitrile (10 mL) and the solvent removed under reduced pressure. Compound 4 is isolated in a pure form using column chromatography.

Biological Data

A. Blood Brain Barrier (BBB) Permeability

Culturing of CACO-2 cells and Determination of Apparent Permeability (Papp) Values:

CACO-2 cells (ATCC number HTB-37) derived from colorectal adenocarcinoma in a 72 year old male were initially cultured in 75 Gm² cell culture flasks (Costar 3376) until confluent CACO-2 cells were grown in EMEM (Sigma 4526) containing 10% FCS, 10 μg/ml insulin (HYBRI-MAX, Sigma 1-4011), non-essential amino acids (Sigma, M7145), glutamine, 50 Uml⁻¹ penicillin and 50 μgml⁻¹ streptomycin (Sigma, P0906). All cells were incubated at 37C in 95% air/5% CO₂. At confluence the cells were used to seed 12 mmTranswell-Col inserts (Costar 3493).

The seeding of 12 mm Transwells was as follows for CACO-2. Flasks of confluent cultures were trypsinsed and cells were carefully resuspended, making sure there are no clumps or air bubbles. 1.5 ml of tissue culture medium was placed in the bottom (acceptor) chamber of the wells and 0.5 ml containing 2×10⁵ cells in the Transwell (donor chamber) and placed in the incubator. The cells were routinely monitored for adequate trans epithelial electrical resistance (TEER) using an EndOhm Tissue resistance measurement chamber (WPI). The subsequent maintenance and feeding of the cells on the Transwells was as follows: when feeding the wells, the medium was removed from the acceptor chamber (basolateral side) and donor chamber (apical side) of the Transwells. The medium was aspirated off with a pipette connected to a vacuum pump, being careful not to touch the filter. 0.5 ml of growth medium was then placed into the donor chamber and 1.5 ml of growth medium was placed into the acceptor chamber.

When the TEER values of the CACO-2 cell monolayers in Transwells was around 500 Ωcm² (300-800 Ωcm² was considered acceptable) permeability experiments were performed in triplicate as follows. All culture media was removed and acceptor and donor chambers were rinsed twice with Eagles Balance Salt Solution (EBSS) (Gibco) at 37° C. 1.5 ml EBSS was added to the acceptor chamber and 0.5 ml EBSS containing radiolabelled compound was added to the donor chamber. Cultures in Transwells were then incubated for 30 minutes at 37° C. at 200 rpm using a Labnet Vortemp. After 30 minutes, 100 μl aliquot was taken from the donor chamber and 750 μl from the acceptor chamber. These aliquots were then counted for radioactivity. The remaining EBSS was removed from the acceptor and donor chambers and then the Transwell was thoroughly rinsed three times la with EBSS. Next, the Transwell membrane (and associated cells) was removed using a scalpel and the amount of radiolabel associated with it was determined.

Permeability of compounds were determined by calculating their Papp value:
Papp=ΔQ/Δt.60.A.Co (cm/sec)

Where, ΔC/Δt is the permeability rate (μg/min); Co is the initial concentration of radiolabelled compound; A is the surface area of membrane. The amount of labelled compound present could be determined from the specific activity of the compound (74 TBq ¹²⁵I/mmol).

Results and Discussion

Permeability through the BBB can be either by passive diffusion which requires a compound being small (<500 Da) and Lipophilic. In this assay, permeable compounds have a Papp value of more than 1×10⁻⁵. For example, as Table 1 shows, ¹⁴C-glucose (Amersham Biosciences) which relies on active transport has a Papp of 5.79×10⁻⁵, and ¹⁴C-diazepam (Amersham Biosciences) which relies on being small and lipophilic has a Papp of 2.44×10⁻⁵. ¹⁴C-Sucrose (Amersham Biosciences) and ¹⁴C-mannitol (Amersham Biosciences) on the other hand, have Papp values of 4.08×10⁻⁶ and 3.63×10⁻⁶ respectively, showing that charge is a contributor to impermeability as both these molecules are small. Compound 1 has a Papp value of 1.37×10⁻⁵ implying that it is permeable through the CACO-2 cell BBB model used. Compound 1 is relatively lipophilic (LogP of 1.75) and small (462.34 Da) and is not prone to H-bond formation (ΔLogP value of −0.38) which also is a benefit when crossing the CACO-2 cell barrier or the BBB. These data suggest that Compound 1 passes through the cell barrier by passive diffusion.

TABLE 1

| Compound | Papp (cm sec⁻¹) |
|---|---|
| Compound 1 | 1.37 × 10⁻⁵ |
| ¹⁴C-Glucose | 5.79 × 10⁻⁵ |
| ¹⁴C-diazapam | 2.44 × 10⁻⁵ |
| ¹⁴C-sucrose | 4.08 × 10⁻⁶ |
| ¹⁴C-mannitol | 3.63 × 10⁻⁶ |

B. Brain Uptake Index (BUI)
Method

The method used is that used by Cornford et al “Metaphalan penetration of the blood-brain barrier via the neutral amino acid transporter in tumour bearing brain.” Cancer Res 52 p138-143 (1992) and involves the injection of a bolus of activity directly into the carotid artery. The animals are decapitated after 15 seconds, the brain removed and the uptake of the test compound calculated with reference to a freely diffusable standard (¹⁴C-Butanol, Amersham Biosciences). BUI is calculated according to the following equation:

$$BUI ( \% ) = \frac{cpm {brain}_{( test )} / cpm {brain}_{( standard )}}{cpm {injectate}_{( test )} / cpm {injectate}_{( standard )}} \times 100 \%$$

BUI was performed on three animals from the same test solution for each experiment. Each compound was assayed in duplicate.

Ceretec™ and Datscan™ were obtained from Amersham Health, ¹⁴C-FDG and ¹⁴C-sucrose were obtained from Amersham Biosciences.

Results

| Compound | Mean BUI | S.D |
|---|---|---|
| Ceretec | 107.25 | 21.02 |
| Datscan | 73.59 | 26.50 |
| Compound 1 | 43.6 | 16 |
| ¹⁴C-FDG | 17.98 | 4.65 |
| ¹⁴C-Sucrose | 0.54 | 0.36 |

Conclusion:

It can be seen that the BUI of Compound 1 is comparable to other compounds that cross the blood-brain barrier. The cut off between compounds that are of low CNS penetration and those that show medium BBB penetration is 20%. It is felt that a value of greater than 20% is acceptable BBB penetration for an imaging agent. Compound 1 therefore shows medium to high BBB penetration, indicative of adequate delivery for a diagnostic of Alzheimer's Disease.

In Situ Brain Perfusion

Method

Compounds may penetrate the brain slowly or be subject to peripheral metabolism, meaning that the BUI may not always be reflective of BBB permeability. For this reason an in situ brain perfusion technique was used. This technique has been extensively used to evaluate the BBB permeability of compounds. The method used is described by Williams et al “Passage of a delta-opioid receptor selective enkephalin, [D-penicillamine2,5] enkephalin, across the blood-brain and the blood-cerebrospinal fluid barriers.” J Neurochem. 1996 March,66(3):1289-99.

The brain was perfused with a saline-based solution containing the test compound for 2, 5, 15, 20 and 30 minutes and uptake in the brain calculated as a percentage of concentration of test substance in the perfusate (R tissue=dpm per g brain/dpm per μl perfusate×100%).

Uptake is plotted against time and the slope of the graph represents the unidirectional brain influx constant, Kᵢₙ. This reflects the rate of entry of a compound into the CNS.

Results

| Compound | Kin (ml/min/g) |
|---|---|
| ¹⁴C-Sucrose | 0.7 |
| Ceretec | 19.8 |
| Compound 1 | 49.8 |

Conclusion

Compound 1 gives a high Kᵢₙ value. This implies that the compound rapidly penetrates the CNS, and backs up the high BUI value.

Capillary Depletion

Compounds that may appear to cross the blood-brain barrier may be sticking to the vasculature within the brain, rather than entering the brain parenchyma. For this reason a capillary depletion method was employed that separates the capillaries from brain homogenate.

Method:

Adapted from Triguero D et al; J Neurochem 1990 54(6):1882-8 “Capillary depletion method for quantification of blood-brain barrier transport of circulating peptides and plasma proteins.” And Thomas nee Williams S A, Segal M B. “Identification of a saturable uptake system for deoxyribonucleosides at the blood-brain and blood-cerebrospinal fluid barriers.” Brain Res. 1996 November 25;741(1-2):230-9.

Male Wistar rats weighing 150-250 g were deeply anaesthetised by the administration of 55 mg/kg sodium pentabarbital (Rhone-Meriaux) by intra peritoneal injection. The left common carotid artery was exposed by blunt dissection, and 100 μl of test solution injected in a single bolus into the carotid using a 30G needle and 1ml syringe (injection time ˜1 second). The injectate contained both the test/validation compound and ¹⁴C— or ³H-Sucrose (Amersham Biosciences) as a non-diffusable standard. Ten seconds after injection, the animal was decapitated and the brain removed. The cerebellum was removed, and the cortex dissected into the left and right hemispheres. Both hemispheres were weighed before homogenisation in 3.5 times their weight of Hanks Buffered Salt Solution (HBSS) using 10-15 strokes using a glass homogeniser. 4 times the brain weight of 26% Dextran solution (73000 molecular weight dextran in HBSS) was added, and the brain further homogenised using 3-5 strokes with the glass homogeniser. All homogenisation periods were performed at 4° C. and were completed within 1 minute. The homogenates were centrifuged for 15 minutes at 5400 g at 4° C. in a refrigerated centrifuge. The vascular enriched pellet and supernatant were then carefully separated. Pellet, supernatant and a sample of injection solution were counted on a Rackbeta Excel scintillation counter, after the addition of 10 ml Hionic Fluor scintillant (Packard). ¹⁴C/³H-Sucrose served to correct for vascular volume of vessels contained in the pellet, and also activity that had leaked from the vasculature into the supernatant during the homogenisation procedure.

Volumes of distribution for pellet and supernatant were calculated as shown below. Data is expressed as ratio of volume of distribution in the supernatant to pellet.

$${V}_{D} = \frac{DPM test ( tissue )}{DPM test ( tissue )} - \frac{DPM sucrose ( injection solution )}{DPM sucrose ( injection solution )}$$

Results

| | Ratio of V_{D} |
|---|---|
| Compound | supernatant/pellet |
| ¹⁴C-Butanol | 51.0 |
| Compound 1 | 7.2 |

Conclusion:

The results imply that Compound 1 is mainly contained within the brain parenchyma. A proportion may be contained within the vasculature (the ratio of supernatant to pellet is not as high as a freely diffusable compound such as ¹⁴C-butanol).

From this it can be concluded that Compound 1 is crossing the blood-brain barrier to enter the brain parenchyma, where it would be able to interact with Alzheimer's Disease pathology. The amount of radiolabel crossing the blood-brain barrier is sufficient to bind and image Alzheimer's pathology

C. Amyloid Binding

Determination of Amyloid Binding of Compound 1:

The binding of Compound 1 (74 TBq/mmol) was determined compared to the ability of ¹²⁵I-beta amyloid protein 1-40 (¹²⁵I-BAP 1-40, Amersham Biosciences IM294)) to bind to amyloid 140 fibrils. Amyloid binding was essentially performed as follows.

Three fresh buffer stocks were prepared for experiments: Buffer 1, 50 mM HEPES/0.1 % Bovine Serum Albumin (BSA) pH 7.5; Buffer 2, 50 mM HEPES/0.1% BSA/400 μM ZnCl₂ pH 7.5; Buffer 3, 50 mM HEPES/0.1% BSA/100 μM ZnCl₂ pH 7.5.

Streptavidin coated scintillation proximity assay beads (SA-SPA beads, Amersham Biosciences) were used to immobilise fibrillar Beta-Amyloid Protein (BAP 1-40). Amyloid coated beads (SPA-BAP) were prepared by incubating 250 μl SA-SPA beads (100 mg/ml) with 250 μl Buffer 2, 425 μl Buffer 1, 50 μl biotinylated BAP 1-40 (0.5 mg/ml, Biosource 03-243), 25 μl BAP 1-40 (10 mg/ml, Biosource 03-138). Non-specific binding SPA beads (SPA-NSB) were prepared to assess the binding of compounds to SPA beads with no associated BAP 1-40 fibrils in the following incubation: 250 μl SA-SPA beads (100 mg/ml) with 250 μL Buffer 2,500 μl Buffer 1.

SPA-BAP and SPA-NSB incubations were left for 24 hours at room temperature and then spun 1.5 ml tubes (eppendorf, Merk, 306/0421/12) for 2 minutes at 1000×g. The supernatants were removed and the beads were washed twice by resuspending them in 1 ml Buffer 3 followed by centrifugation for 2 minutes at 1000×g. Finally, washed SPA-BAP and SPA-NSB beads were resuspended in 1 ml Buffer 3.

Amyloid binding of ¹²⁵I-BAP 1-40 and Compound 1 was performed in triplicate in 0.5 ml tubes (eppendorf, Merk, 306/0421/02) by adding 50 μl SPA-BAP beads to 25 μl Buffer 2 and 25 μl labelled compound (¹²⁵I-BAP 1-40 or Compound 1). Tubes were then incubated for 180 minutes at room temperature with shaking, followed by centrifugation for 2 minutes at 1000×g. The supernatants were removed and SPA-BAP pellet washed twice with 300 μl Buffer 3 containing 1% TWEEN-20 (Sigma, P7949). Non-specific binding for labelled compounds to the SPA beads was determined using incubations as described above but by substituting SPA-BAP beads with SPA-NSB beads. Radioactivity associated with the washed SPA bead pellets was then determined.

The affinity of labelled compounds for fibrillar BAP 1-40 was estimated by subtracting SPA-NSB associated counts from SPA-BAP associated counts. The binding of labelled compounds was then compared to ¹²⁵I-BAP binding, which was taken as being 100%.

In these experiments, ¹²⁵-BAP 1-40 or Compound 1 were added in equimolar amounts (between 1-5×10⁻¹¹ mmoles per incubation).

Results and Discussion

BAP 1-40 readily self-aggregates. In this assay, the binding of ¹²⁵I-BAP 1-40 to a fixed amount of amyloid fibrils immobilised on SPA beads was used as a reference for other compounds. Table 1 shows how other amyloid binders and non-binders compare to the binding of ¹²⁵I-BAP 1-40. Compound 1 binds to amyloid fibrils with 27% affinity of ¹²⁵I-BAP 1-40, which is favourable compared to the ¹²⁵I-labelled BAP 15-21 sequence (Amersham Biosciences) (21%) and the ^{99m}Tc-labelled BAP 15-21 sequence (9%). The BAP 15-21 sequence is responsible for the binding of BAP to itself during the formation of amyloid fibils.

TABLE 1

| | Amyloid binding |
|---|---|
| Compound | (% of 125I-BAP 1-40) |
| ¹²⁵I-BAP 1-40 | 100 |
| Compound 1 | 27 |
| ¹²⁵I-KKLVFFA (BAP 15-21) | 21 |
| ^{99m}Tc-Pn216-KKLVFF (BAP 15-20) | 9 |

D. Pharmacokinetics
Materials and Method

Compound 1 IMQ1961 prepared by Amersham Biosciences 250 μCi/ml (specific activity 2000Ci/mmol) freshly diluted to give 1 μCi/0.1 ml volume injection bolus. 15 normal male wistar rats 150-180 g (Charles River).

Male wistar rats were anaethetised briefly prior to injection with Compound 1 (1 μCi, 0.1 ml bolus tail vein). Biodistributions were performed at five time points (5, 15, 30, 60 and 120 mins) in triplicate animals in which tissues from brain, blood, muscle, kidney, spleen, stomach, small intestines, large intestines and faeces, bladder and urine, fat, skin and thyroid were dissected and counted for distribution of the compound in the body in a Wallac Wizzard gamma counter.

Percentage injected dose calculations were made and plotted using the Qk pharmacokinetics program to determine t½ α, and volume of distribution (V_{D}). Relative retention (RR) and Brain:tissue ratios were calculated as follows;

$$Relative retention = \frac{cpm brain / brain weight ( g )}{cpm remaining in body / body weight ( g )}$$

$$Brain \text{:} Blood ratio = \frac{cpm brain / brain weight ( g )}{cpm blood / blood weight ( g )}$$

Results and Discussion

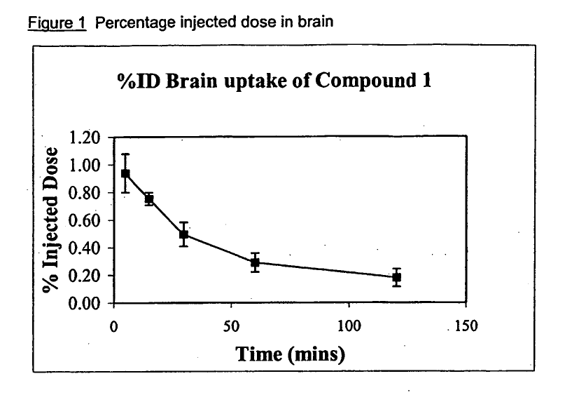

Biodistribution data are shown for Compound 1 (Table 2 and FIG. 1 ). Data show percentage injected dose in the brain in normal wistar rats changes over the two hour experiment. Initial delivery is 0.94% to the brain which clears out with a t½ values of 12.4 min (α) to yield an uptake of 0.29% at 60 mins. Low thyroid uptake (<1%) indicates that the iodinated compound is very stable in vivo and does not degrade to iodide as often the case with other iodinated molecules.

The volume of distribution of the compound is high (2.27×10⁴ L/kg) which is expected for a lipophilic compound such as Compound 1. Compound 1 Log P and delta LogP values of 1.75 and −0.38, respectively facilitate its transport through plasma membranes. Consequently high % ID values in fat tissue and to a lesser extent, skin, are observed.

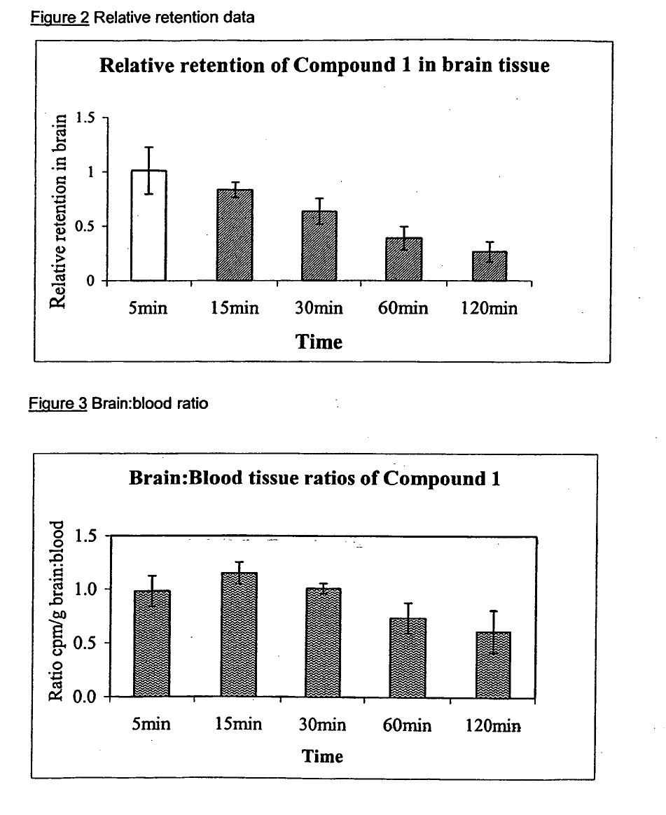

Relative retention of the compound is the retention in the brain tissue with respect to the rest of the body. FIG. 2 shows relative retention for Compound 1 is 1.01 initially at 5 minutes, compared to blood that has a similar 1.04. Decreases in relative retention with time suggest Compound 1 is taken up maximally within five minutes and is then cleared more quickly than clearance through the rest of the tissues in the body. Brain:blood ratios (FIG. 3) indicate Compound 1 clears from blood faster than brain between 15 and 30 mins. Clearance through the body is largely via the hepatobiliary system, in keeping with the pharmacokinetics profile for a lipophilic compound and little is via the urinary system.

TABLE 2

| | | 5 min | | 15 min | | 30 min | | 60 min | | 120 min |
|---|---|---|---|---|---|---|---|---|---|---|
| | 5 min | SD | 15 min | SD | 30 min | SD | 60 min | SD | 120 min | SD |
| Muscle | 21.49 | 4.00 | 19.83 | 1.71 | 12.59 | 2.45 | 7.79 | 2.17 | 5.60 | 0.68 |
| Blood | 6.05 | 1.58 | 4.14 | 0.32 | 3.58 | 0.59 | 2.77 | 0.35 | 2.27 | 0.58 |
| Kidney | 3.14 | 0.65 | 1.60 | 0.22 | 0.95 | 0.22 | 0.63 | 0.15 | 0.45 | 0.14 |
| urine | 0.04 | 0.01 | 2.10 | 2.90 | 1.63 | 0.37 | 8.39 | 1.96 | 10.02 | 1.06 |
| lung | 1.40 | 0.26 | 0.49 | 0.43 | 0.48 | 0.04 | 0.34 | 0.11 | 0.23 | 0.04 |
| liver | 19.47 | 4.01 | 7.48 | 2.24 | 4.46 | 0.76 | 3.12 | 0.40 | 2.39 | 0.66 |
| Spleen | 0.69 | 0.34 | 0.32 | 0.07 | 0.19 | 0.05 | 0.15 | 0.05 | 0.08 | 0.01 |
| Stomach | 1.82 | 1.12 | 2.56 | 1.00 | 6.66 | 1.60 | 8.98 | 1.01 | 9.29 | 3.27 |
| SI | 9.04 | 0.37 | 12.60 | 1.83 | 21.93 | 2.56 | 28.42 | 10.16 | 26.86 | 6.27 |
| LI | 1.53 | 0.77 | 1.77 | 1.26 | 1.90 | 1.15 | 7.75 | 11.99 | 3.12 | 3.16 |
| Brain | 0.94 | 0.14 | 0.75 | 0.05 | 0.49 | 0.09 | 0.29 | 0.07 | 0.18 | 0.06 |
| thyroid | 0.16 | 0.14 | 0.16 | 0.12 | 0.39 | 0.53 | 0.17 | 0.20 | 0.95 | 0.52 |
| Skin | 8.92 | 2.20 | 12.80 | 0.33 | 16.90 | 1.77 | 17.14 | 3.37 | 12.89 | 1.05 |
| Fat | 30.82 | 15.61 | 36.31 | 2.86 | 59.02 | 13.66 | 73.92 | 9.36 | 55.62 | 21.14 |
| inj site | 2.12 | 0.64 | 1.86 | 0.58 | 3.05 | 2.69 | 1.83 | 0.49 | 2.62 | 1.59 |

1. Use of a compound of formula (I): 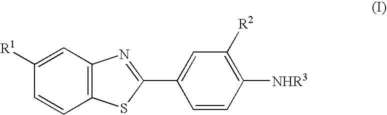
or a salt thereof, wherein:
R¹ is ¹²⁵I, ¹²⁴I, ¹²³I, ⁷⁵Br, ⁷⁶Br, ¹⁸F;
R² is C₁₋₆ alkyl; and
R³ is selected from hydrogen, C₁₋₆ alkyl, —C(O)C₁₋₆ alkyl, —C(O)C₁₋₆ haloalkyl, and —C(O)CH(R⁴)NH₂;
wherein R⁴ is selected from hydrogen, C₁₋₆alkyl, C₁₋₆hydroxyalkyl, and C₁₋₆aminoalkyl;
for the manufacture of a radiopharmaceutical for the in vivo diagnosis or imaging of an amyloid-associated disease, particularly Alzheimer's disease.

2. Use according to claim 1 of a compound of formula (I): 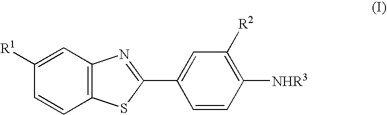
or a salt thereof, wherein:
R¹ is ¹²⁵I, ¹²⁴I, ¹²³I, ⁷⁵Br, ⁷⁶Br, or ¹⁸F;
R² is C₁₋₆ alkyl; and
R³ is selected from hydrogen, C₁₋₆ alkyl, —C(O)C₁₋₆ alkyl, and —C(O)C₁₋₆ haloalkyl;
for the manufacture of a radiopharmaceutical for the in vivo diagnosis or imaging of an amyloid-associated disease, particularly Alzheimer's disease.

3. Use according to claim 1, where in the compound of formula (I):
R¹ is ¹²⁵I, ¹²⁴I, ¹²³I, or ¹⁸F;
R² is methyl; and
R³ is selected from hydrogen and —C(O)C₁₋₆ haloalkyl.

4. Use according to claim 1, wherein the compound of formula (I) is selected from:
5-[¹²⁵I]-iodo-2-(4′-amino-3′-methylphenyl)benzothiazole;
5-[¹²⁴I]-iodo-2-(4′-amino-3′-methylphenyl)benzothiazole;
5-[¹²³I]-iodo-2-(4′-amino-3′-methylphenyl)benzothiazole;
5-[¹²⁵I]-iodo-2-(4′-trifluoromethylamido-3′-methylphenyl)benzothiazole;
5-[¹²⁴I]-iodo-2-(4′-trifluoromethylamido-3′-methylphenyl)benzothiazole;
5-[¹²³I]-iodo-2-(4′-trifluoromethylamido-3′-methylphenyl)benzothiazole;
5-[¹⁸F]-fluoro-2-(4′-amino-3′-methylphenyl)benzothiazole; and
5-[¹⁸F]-fluoro-2-(4′-trifluoromethylamido-3′-methylphenyl)benzothiazole.

5. A method for the in vivo diagnosis or imaging of amyloid-associated disease in a subject comprising administration of a compound of formula (I) or a salt thereof as defined in claim 1.

6. A radiopharmaceutical formulation which comprises a compound of formula (I) or a salt thereof as defined in claim 1.

7. A compound of formula (Ia): 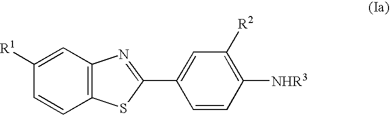
or a salt thereof, wherein:
R¹ is ¹²⁵I, ¹²⁴I, ¹²³I, ⁷⁵Br, or ⁷⁶Br;
R² is C₁₋₆ alkyl; and
R³ is selected from hydrogen, C₁₋₆ alkyl, —C(O)C₁₋₆ alkyl, and —C(O)C₁₋₆ haloalkyl.

8. A compound of formula (Ia) according to claim 7 wherein:
R¹ is ¹²⁵I, ¹²⁴I, or ¹²³I;
R² is methyl; and
R³ is selected from hydrogen and —C(O)C₁₋₆ haloalkyl.

9. A compound of formula (Ia) according to claim 7 wherein the compound of formula (I) is selected from:
5-[¹²⁵I]-iodo-2-(4′-amino-3′-methylphenyl)benzothiazole;
5-[¹²⁴I]-iodo-2-(4′-amino-3′-methylphenyl)benzothiazole;
5-[¹²³I]-iodo-2-(4′-amino-3′-methylphenyl)benzothiazole;
5-[¹²⁵I]-iodo-2-(4′-trifluoromethylamido-3′-methylphenyl)benzothiazole;
5-[¹²⁴I]-iodo-2-(4′-trifluoromethylamido-3′-methylphenyl)benzothiazole; and
5-[¹²³I]-iodo-2-(4′-trifluoromethylamido-3′-methylphenyl)benzothiazole.
